(19) United States
(12) Patent Application Publication
(10) Pub. No.: US 2008/0260656 A1
(43) Pub. Date: Oct. 23, 2008

(54) Ungual/periungual compositions comprising morpholine compounds and water-soluble film-forming agents

(75) Inventor: Claire Mallard, Mougins (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 12/081,207

(22) Filed: Apr. 11, 2008

Related U.S. Application Data
(63) Continuation of application No. PCT/FR2006/002307, filed on Oct. 13, 2006.

(30) Foreign Application Priority Data
Oct. 14, 2005 (FR) 0510504

(51) Int. Cl. A61K 31/5377; A61P 31/10; A61K 9/12; A61K 8/49; A61Q 3/02

(52) U.S. Cl. 424/45; 514/239.5; 424/61

(57) ABSTRACT

Pharmaceutical compositions suited for ungual and periungual application and useful for the treatment of dermatological conditions/afflictions, in particular onychomycosis, contain an anti-mycotic agent of the family of the morpholines and a water-soluble film-forming agent.

### CROSS-REFERENCE TO PRIORITY/PCT APPLICATIONS

This application claims priority under 35 U.S.C. § 119 of FR 0510504, filed Oct. 14, 2005, and is a continuation of PCT/FR 2006/002307, filed Oct. 13, 2006 and designating the United States (published in the French language on Apr. 19, 2007 as WO 2007/042682 A1; the title and abstract were also published in English), each hereby expressly incorporated by reference in its entirety and each assigned to the assignee hereof.

### BACKGROUND OF THE INVENTION

1. Technical Field of the Invention

The present invention relates to compositions for ungual and periungual administration, useful for the treatment of dermatological conditions or afflictions, in particular onychomycosis, which comprise an anti-mycotic agent of the family of the morpholines and a water-soluble film-forming agent.

2. Description of Background and/or Related and/or Prior Art

The nails can be subjected to disorders, deficiencies or pathologies of varied nature and origin (Baran R. et al., Diseases of the nails and their management. 3rd Edition. 2001). Exemplary thereof is paronychia, which can be caused by bacteria, fungi, parasites or viruses, which can derive from dermatological or systemic diseases or else which can originate from a drug treatment.

The fungal pathologies can be restricted specifically to the nail, such as onychomycosis, or else can, like herpes or syphilis, affect other parts of the body; they can also have an effect on the physiology of the nail. Fungal infections of the nails are frequently caused by dermatophytes but can also be caused by moulds, fungi and/or yeasts.

The treatments employed today are either local treatments or general treatments; the two are often combined for optimal effectiveness. This is because a treatment, to be effective, has to be lengthy, in order to match the time for regrowth of a nail. Furthermore, the mycosis may be located in the nail or in the bed of the nail, which requires that the active agent penetrate the nail in its entirety.

General treatments often elicit harmful side effects and topical treatments are often less effective only because of the difficult penetration of the active principles through the nail.

Film-forming solutions or nail varnishes are currently more particularly used for the treatment of onychomycosis and similar fungal infections of the nails in human beings or mammals.

Numerous compositions comprising active agents having an anti-fungal activity are described in the literature for the prevention and treatment of these conditions. For example, U.S. Pat. No. 6,319,509 describes a pharmaceutical composition in the form of a nail varnish useful for the treatment of dermatological conditions which comprises terbinafine and a film-forming agent of water-insoluble type. U.S. Pat. No. 6,495,124 describes a pharmaceutical composition useful for the treatment of conditions of the nails which comprises an anti-mycotic active agent, a plasticizing agent and an absorption promoter, all in the form of a varnish comprising a volatile solvent and a water-insoluble polymer. WO 2004/084826, for its part, describes a pharmaceutical composition in the form of a varnish which comprises an anti-fungal agent of the allylamine and azole family, an absorption promoter and a water-soluble polymer.

The assignee hereof has also described, in FR-2,844,197, a combination of propenetrating agents acting synergistically for the preparation of a solution for ungual and periungual application for dermatological or cosmetic use and the solution resulting therefrom.

The effectiveness of a nail varnish as delivery vehicle for a topical application of an active agent has been described by Marty in J. Eur. Acad. Dermatol. Venerol., 4 (suppl. 1), pp. 17-21 (1995). This concerns the study of the delivery of an anti-fungal agent, amorolfine chlorohydride (or amorolfine HCl). The base combination of a varnish, composed of a solvent, a plasticizer and a water-insoluble film-forming agent, as described in the literature, unfortunately does not make possible optimal penetration of the active agent into the nail. This is because such a combination is the cause in particular of a phenomenon of limitation of the diffusion of the active agent.

Furthermore, the use of a film-forming agent of water-insoluble type in conventional varnish compositions, such as those described in the prior art, promotes an excessively rapid rate of drying (less than one minute) and tends to provoke, in certain sensitive subjects, irritation of the skin surrounding the nail.

Essential need continues to exist to develop a dermatological or cosmetic composition of film-forming solution type for ungual and periungual application which makes possible better penetration of the active principles through the nail, for this reason rendering the active agents more effective, without causing irritation of the skin, which contributes to a reduction in the treatment time.

### SUMMARY OF THE INVENTION

It has now unexpectedly been discovered that the formulation of water-soluble polymers into a composition of film-forming solution type makes it possible to obtain better bioavailability of the active agent within the nail by virtue of more effective diffusion of the latter from the film towards the nail. In addition, the formulation of a water-soluble film-forming agent in the composition of a varnish makes possible the formation of a softer and less brittle film than that obtained with varnishes of conventional composition, namely, with a water-insoluble film-forming agent.

The present invention thus features pharmaceutical compositions, in particular for ungual and periungual application, useful for the treatment of dermatological conditions/afflictions, which comprise:

an anti-mycotic active agent selected from the family of the morpholines and derivatives thereof;

a water-soluble film-forming agent, with the exception of hydroxyalkylchitosans and carboxyalkylchitosans;

at least one absorption promoter; and

an organic solvent or a mixture of organic solvents/cosolvents.

### DETAILED DESCRIPTION OF BEST MODE AND SPECIFIC/PREFERRED EMBODIMENTS OF THE INVENTION

Preferably, the anti-mycotic active agent is selected from the family of the morpholines and its derivatives, such as, for example, fenpropimorph or tridemorph. Preferably, the anti-mycotic agent will be amorolfine or one of its pharmaceutically acceptable salts.

The term “amorolfine” means amorolfine base.

The term “pharmaceutically acceptable salts” means salts compatible with the skin, mucous membranes and/or superficial body growths and in particular the salts formed with physiologically acceptable organic or inorganic acids. Exemplary of these are hydrohalic acids, such as hydrobromic acid or hydrochloric acid, or also phosphoric acid or nitric acid, or also mono- and bifunctional carboxylic and hydroxycarboxylic acids, such as acetic acid, maleic acid, succinic acid, fumaric acid, tartaric acid, citric acid, salicylic acid, sorbic acid and lactic acid, and, lastly, sulfonic acids, such as 1,5-naphthalenedisulfonic acid.

Preferably, hydrochloric acid will be used to form amorolfine hydrochloride. This is because amorolfine hydrochloride exerts a fungistatic and fungicidal activity by inhibition of the synthesis of the sterols of the cell membrane of fungi, such as yeasts, dermatophytes, moulds and Dematiaceae (black fungi).

In a preferred embodiment, the present invention also features compositions for ungual and periungual application, useful for the treatment of dermatological conditions/afflictions, which comprise:

amorolfine hydrochloride as anti-mycotic agent;

a water-soluble film-forming agent;

at least one absorption promoter;

an organic solvent or a mixture of organic solvents/cosolvents; and,

optionally, a plasticizing agent.

Preferably, the concentration of anti-mycotic active agent, in particular of amorolfine hydrochloric, ranges from 0.01 to 20% by weight, with respect to the total weight of the composition (w/w), and more particularly from 0.1 to 8% and preferably equal to 2.5%, 3.5% or 5% by total weight, with respect to the total weight of the composition.

The term “pharmaceutical composition for ungual and periungual application” in particular means a nail varnish or film-forming solution for application to the nails and their periphery.

The compositions according to the invention comprise a water-soluble film-forming agent. The term “film-forming agent” defines a bonding agent which makes possible the formation of a film or also of a layer. The term “water-soluble film-forming agent” means a film-forming agent which is completely compatible with water, such that, at a temperature of 20° C., one gram of film-forming agent is soluble in 100 grams of water, preferably in 50 grams or less, or also in 30 grams or less, and more preferably still in 10 grams of water or less.

The water-soluble film-forming agent is advantageously selected from among polyvinylpyrrolidones and derivatives thereof, polysaccharides, gums, polyvinyl alcohols, celluloses and derivatives thereof, cyanoacrylic polymers, or also acrylic polymers and copolymers and polyacrylamides which are soluble in water. The water-soluble film-forming agents according to the invention can be of natural origin, such as chitosans, with the exception of hydroxyalkylchitosans and carboxyalkylchitosans.

Among the polyvinylpyrrolidones and derivatives thereof, exemplary are poly(1-vinyl-2-pyrrolidone), vinylpyrrolidone/vinyl acetate copolymer and vinylpyrrolidone/dimethylaminoethyl methacrylate copolymer.

Exemplary polysaccharides are the celluloses and derivatives thereof, such as carboxymethylcellulose or also hydroxypropylcellulose or hydroxyethylcellulose. Also exemplary polysaccharides are sodium hyaluronates or pectins.

Exemplary gums are guar gum, carrageenan gums, karaya gum or xanthan gum. And also exemplary are polyacrylamides or acrylic/methacrylic, polymethacrylate/butyl acrylate or acrylic/acrylate copolymers.

Preferably, the water-soluble film-forming agent according to the invention is selected from polyvinylpyrrolidones and their soluble copolymers, such as, for example, copovidone; alternatively, the water-soluble film-forming agent according to the invention is selected from celluloses and derivatives thereof, polysaccharides and cyanoacrylic polymers.

The film-forming agent as described above is used at preferred concentrations ranging from 0.01 to 50% w/w, preferably 0.5 to 30% w/w.

In addition, according to the invention, the pharmaceutical composition comprises at least one promoter of absorption into the nail.

The term “promoter of absorption into the nail” means a pharmaceutically acceptable chemical compound capable of increasing the permeability of the nail with regard to the active principles, so as to enhance the kinetics of penetration of these active principles through the nail.

Generally, the absorption promoters used can be selected from the following list, without this being limited with regard to the scope of the invention: sulfoxides, fatty acids, fatty acid esters, polyol ethers and polyols, amides, surfactants, terpenes, alkanones, lactones, mercaptans, aliphatic organic compounds, amino acids and derivatives, dioxolanes, azone, and mixtures thereof.

Exemplary sulfoxides are dimethyl sulfoxide (DMSO), decylmethyl sulfoxide, and mixtures thereof.

Exemplary fatty acids are valeric, heptanoic, pelargonic, caproic, capric, lauric, myristic, stearic, oleic, linoleic, linolenic, caprylic, isovaleric, neopentanoic, neoheptanoic, neononanoic, trimethylhexanoic, neodecanoic or isostearic acids, and mixtures thereof.

Exemplary fatty acid esters are isopropyl n-butyrate, isopropyl n-hexanoate, isopropyl n-decanoate, isopropyl myristate, isopropyl palmitate, octyidodecyl myristate, ethyl acetate, butyl acetate, methyl acetate, methyl valerate, methyl propionate, diethyl sebacate, ethyl oleate, ethyl laurate, and mixtures thereof.

Exemplar polyol ethers include, without this list being limiting, ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether, polypropylene glycol, polyethylene glycol, polyethylene glycol dodecyl ether, diethylene glycol monoethyl ether, polyethylene glycol-8 glycerol caprylate, and mixtures thereof.

Examples of polyols, without this list being limiting, are ethylene glycol, propylene glycol, butylene glycol, hexylene glycol, propylene glycol monocaprylate, and mixtures thereof.

Examples of amides are urea, dimethylacetamide, diethyltoluamide, dimethylformamide, dimethyloctamide, dimethyldecamide, 1-alkyl-4-imidazolin-2-one, cyclic amides, hexamethylene lauramide and derivatives thereof, diethanolamine, triethanolamine, pyrrolidone derivatives, such as 1-methyl-2-pyrrolidone, 2-pyrrolidone, 1-lauryl-2-pyrrolidone, 1-lauryl-4-carboxy-2-pyrrolidone or 1-methyl-4-carboxy-2-pyrrolidone, and mixtures thereof.

Exemplary surfactants which can be used according to the invention, are anionic surfactants, cationic surfactants, non-ionic surfactants, amphoteric surfactants, bile salts and lecithin.

Exemplary anionic surfactants include sodium laurate, sodium lauryl sulfate, and mixtures thereof.

Exemplary cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride or hexadecyltrimethylammonium chloride.

Exemplary non-ionic surfactants, without this list being limiting, include polyoxyethylene ether, polyoxyethylene sorbitan esters, polyethylene glycol fatty alcohol esters, and mixtures thereof.

Exemplary amphoteric surfactants, without this list being limiting, include lauramidopropyl betaine, cocamidopropyl betaine, lauryl betaine, coco betaine, cocamidopropyl hydroxysultaine, aminopropyl laurylglutamide, sodium cocoamphoacetate, sodium lauroamphoacetate, disodium lauroamphodiacetate, disodium cocoamphodiacetate, sodium cocoamphopropionate, disodium lauroamphodipropionate, disodium cocoamphodipropionate, sodium laurimidodipropionate, disodium cocoamphocarboxymethylhydroxypropylsulfonate, and mixtures thereof.

Exemplary terpenes according to the invention include D-limonene, α-pinene, β-enrene, α-terpineol, terpinen-4-ol, carvol, carvone, pulegone, piperitone, menthone, menthol, geraniol, cyclohexene oxide, limonene oxide, α-pinene oxide, cyclopentene oxide, 1,8-cineol, oil of ylang-ylang, oil of anise, oil of eucalyptus, and mixtures thereof.

Among the alkanones, useful are those of n-heptane, n-octane, n-nonane, n-decane, n-undecane, n-dodecane, n-tridecane, n-tetradecane, n-hexadecane, and mixtures thereof.

Exemplary lactones according to the invention are cyclopentadecalactone, marketed under the trademark CPE-125 by Bentley.

An exemplary mercaptan is N-(2-mercaptopropionyl)glycine. Exemplary aliphatic organic compounds are C₁ to C₁₈, preferably C₁ to C₁₂, aliphatic organic mono- or polycarboxylic acids and derivatives thereof, such as, in particular, hydroxymonocarboxylic acids and hydroxydicarboxylic acids.

Exemplary C₁ to C₁₂ aliphatic carboxylic acids include, in particular, hydroxy acids, for example, without implied limitation, methanoic acid, 2-methylbutanoic acid, propanoic acid, 2-methylpropanoic acid, 2,2-dimethylpropanoic acid, decanoic acid, octanoic acid, hex-2-enoic acid, heptanoic acid, 6-methylheptanoic acid, 3-ethylpentanoic acid, 3-chloropentanoic acid, 2-hydroxypropanoic acid, 2-chloro-4-hydroxyhexanoic acid, hexanedioic acid, octadecanoic acid, 4-oxopentanoic acid, 6-hydroxy-4-oxononanoic acid, 2-ketopropanoic acid, tartronic acid, maleic acid, tartaric acid, glucaric acid, citric acid, lactic acid, glycolic acid, isocitric acid, tropic acid, 5-hydroxylauric acid, 3-hydroxy-4-methoxymandelic acid or their mixtures.

Examples of amino acids and derivatives are the amino acids comprising a sulfur atom, such as, without implied limitation, L-cysteine, D-cysteine, D,L-cysteine, N-acetyl-L-cysteine, D,L-homocysteine, L-cystine methyl ester, L-cystine ethyl ester, N-carbamoylcysteine, glutathione and cysteamine.

An example of dioxolane is 2-(n-nonyl)-1,3-dioxolane, also marketed under the trademark SEPA by Macrochem.

Preferably, the absorption promoter is selected from among urea, lactic acid, N-acetyl-L-cysteine and mixtures thereof.

The absorption promoter or promoters as described above are used in proportions which make possible optimum penetration of the active principle. Preferably, the absorption promoter or promoters are present in the composition in concentrations ranging from 0.1 to 20% and more particularly from 0.25 to 10%.

As indicated above, the compositions according to the invention comprise a solvent or else a mixture of a solvent with one or more cosolvents. The presence of a film-forming agent of water-soluble type allows the use of a large number of types of solvents.

The solvents and/or cosolvents can be selected from the family of organic solvents and are Class 3 solvents with a low toxic potential according to the ICH standards (Impurities: Guideline for Residual Solvents, International Conference of Harmonization), such as ethanol, isopropyl alcohol, acetone, methyl acetate, ethyl acetate, butyl acetate, alkyl methyl sulfoxides, 2-propanol, methyl isobutyl ketone, 1-butanol, dichloromethane, N-methyl-2-pyrrolidone or their mixtures.

Among the solvents and/or cosolvents described above, preferably employed are volatile organic solvents and/or cosolvents. Ethanol is a particularly preferred solvent.

Preferably, the solvents and/or cosolvents are included at concentrations of greater than or equal to 60% by weight, with respect to the total weight of the composition, preferably in concentrations ranging from 70 to 90% by weight, with respect to the total weight of the composition.

Moreover, it is possible to use purified water as cosolvent, in concentrations ranging from 0.01 to 30% by weight, with respect to the total weight of the composition.

In addition, the latter can be used in combination with one or more of the solvents and/or cosolvents mentioned above.

The compositions according to the invention can optionally comprise a plasticizing agent. Among the plasticizing agents, without this list being limiting, are compounds such as phthalates, triacetates, citrates or their mixtures.

The plasticizing agent is preferably present at concentrations ranging from 0.01 to 10% by weight, with respect to the total weight of the composition, and more particularly ranging from 0.01 to 5% by weight.

Furthermore, the compositions as described above are preferably provided in the form of sprayable solutions, with or without a propellant gas.

In the case where the composition comprises a propellant gas, it is preferably selected from the group consisting of propane, butane, dichlorodifluoromethane, dichlorotetrafluoroethane, octafluorocyclobutane, nitrogen, carbon dioxide, dimethyl ether and mixtures thereof.

According to a preferred embodiment of the invention, the propellant gas is in the liquid form and its concentration is from 0.01 to 30% by weight, with respect to the total weight of the composition.

Other active agents can be incorporated in addition to the anti-mycotic agent, such as an anti-biotic agent, an anti-bacterial agent, a steroidal anti-inflammatory agent, a non-steroidal anti-inflammatory agent, an anti-parasitic agent, an anti-viral agent, an immunosuppressant agent, an agent which is a modulator of nuclear receptors, or their mixtures.

The solutions for ungual and periungual application, in particular the nail varnish according to the invention, can additionally comprise any additive normally used in the cosmetic or pharmaceutical field, such as sequestering agents, wetting agents, adhesion agents, spreading agents, antioxidants, sunscreens, preservatives, fillers, electrolytes, humectants, pigments, dyes, normal inorganic or organic bases or acids, essential oils, cosmetic active principles, moisturizing agents, vitamins, essential fatty acids or sphingolipids.

Of course, one skilled in this art will take care to choose this or these optional additional compounds and/or their amounts such that the advantageous properties of the compositions according to the invention are not, or not substantially, detrimentally affected.

The pharmaceutical compositions according to the invention are particularly well suited for the following fields of dermatological treatment, whether regime or regimen:

onychomycosis, green nails, paronychia, including acute bacterial paronychia and chronic paronychia, erysipeloid, onychorrexis, gonococcus infection, swimming pool granuloma, larva migrans, leprosy, orf nodule, milkers' nodule, herpetic whitlow, sporotrichosis, syphilis, tuberculosis verrucosa cutis, tularaemia, tungiasis, peri- and subungual warts, herpes zoster, twenty-nail dystrophy (trachyonychia) and dermatological diseases which have an effect on the nails, such as psoriasis, pustular psoriasis, alopecia areata, parakeratosis pustulosa, contact dermatosis, Reiter's syndrome, psoriasiform acral dermatitis, lichen planus, idiophatic atrophy of the nails, lichen nitidus, lichen striatus, inflammatory linear verrucous epidermal naevus (ILVEN), pelade, pemphigus, bullosa pemphigoid, acquired epidermolysis bullosa, Darier's disease, pityriasis rubra pilaris, palmoplantar keratoderma, contact eczema, erythema multiforme, scabies, Basex's syndrome, systemic scleroderma, systemic lupus erythematosus, chronic lupus erythematosus or dermatomyositis.

The present invention also thus relates to the formulation of compositions as medicaments for the treatment and/or the prevention of the pathologies described above.

The present invention also features, in addition, compositions for ungual and periungual application as described above formulated as medicaments useful for the treatment and/or for the prevention of fungal pathologies, preferably onychomycosis.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Composition

| | |
|---|---|
| Amorolfine HCl | 6.4% |
| Urea | 2.5% |
| Lactic acid | 4.0% |
| Polyvinylpyrrolidone | 3.0% |
| Ethanol | q.s.p. for 100% |

### Example 2

### Composition

| | |
|---|---|
| Amorolfine HCl | 6.4% |
| Urea | 2.5% |
| N-Acetyl-L-cysteine | 1.5% |
| Hydroxyethylcellulose | 1.0% |
| Ethanol | q.s.p. for 100% |

### Example 3

### Composition

| | |
|---|---|
| Amorolfine HCl | 6.4% |
| N-Acetyl-L-cysteine | 1.5% |
| Hydroxyethylcellulose | 1.0% |
| Water | 10.0% |
| Ethanol | q.s.p. for 100% |

### Example 4

### Composition

| | |
|---|---|
| Amorolfine HCl | 6.4% |
| Cetearyl alcohol | 2.5% |
| Polyvinylpyrrolidone | 3.0% |
| Ethyl acetate | 4.0% |
| Ethanol | q.s.p. for 100% |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A pharmaceutical/dermatological composition suited for ungual and periungual application, comprising:
at least one morpholino anti-mycotic active agent or derivative thereof;
at least one water-soluble film-forming agent, other than a hydroxyalkylchitosan or carboxyalkylchitosan;
at least one absorption promoter; and
an organic solvent or a mixture of organic solvents/cosolvents.

2. The pharmaceutical/dermatological composition as defined by claim 1, said at least one morpholino anti-mycotic agent comprising fenpropimorph, tridemorph, amorolfine and or pharmaceutically acceptable salts thereof.

3. The pharmaceutical/dermatological composition as defined by claim 1, comprising a pharmaceutically acceptable amorolfine salt formed from an acid selected from among hydrohalic acids, mono- and bifunctional carboxylic and hydroxycarboxylic acids, and sulfonic acids.

4. The pharmaceutical/dermatological composition as defined by claim 3, comprising amorolfine hydrochloride.

5. The pharmaceutical/dermatological composition as defined by claim 4, comprising:
amorolfine hydrochloride as anti-mycotic agent;
at least one water-soluble film-forming agent;
at least one absorption promoter; and
an organic solvent or a mixture of organic solvents/cosolvents.

6. The pharmaceutical/dermatological composition as defined by claim 1, wherein the concentration of anti-mycotic agent ranges from 0.01 to 20% by weight, with respect to the total weight of the composition.

7. The pharmaceutical/dermatological composition as defined by claim 6, wherein the concentration of anti-mycotic agent is 2.5%, 3.5% or 5% by total weight, with respect to the total weight of the composition.

8. The pharmaceutical/dermatological composition as defined by claim 1, comprising at least one water-soluble film-forming agent selected from among polyvinylpyrrolidones and derivatives thereof, polysaccharides, gums, polyvinyl alcohols, celluloses and derivatives thereof, cyanoacrylic polymers, and acrylic polymers and copolymers and polyacrylamides which are soluble in water.

9. The pharmaceutical/dermatological composition as defined by claim 8, comprising at least one film-forming agent selected from among polyvinylpyrrolidones and soluble copolymers thereof.

10. The pharmaceutical/dermatological composition as defined by claim 8, comprising at least one film-forming agent selected from among celluloses and derivatives thereof, polysaccharides and cyanoacrylic polymers.

11. The pharmaceutical/dermatological composition as defined by claim 1, comprising from 0.01 to 50% w/w of said at least one film-forming agent

12. The pharmaceutical/dermatological composition as defined by claim 1, comprising at least one absorption promoter selected from among sulfoxides, fatty acids, fatty acid esters, polyols, amides, surfactants, terpenes, alkanones, lactones, mercaptans, aliphatic organic compounds, amino acids and derivatives thereof, dioxolanes, azone and mixtures thereof.

13. The pharmaceutical/dermatological composition as defined by claim 1 comprising at least one absorption promoter selected from among urea, lactic acid, N-acetyl-L-cysteine and mixtures thereof.

14. The pharmaceutical/dermatological composition as defined by claim 1, comprising from 0.1 to 20% of said at least one absorption promoter.

15. The pharmaceutical/dermatological composition as defined by claim 1, comprising an organic solvent and/or at least one organic cosolvent selected from among ethanol, isopropyl alcohol, acetone, methyl acetate, ethyl acetate, butyl acetate, alkyl methyl sulfoxides, 2-propanol, methyl isobutyl ketone, 1-butanol, dichloromethane, N-methyl-2-pyrrolidone and mixtures thereof.

16. The pharmaceutical/dermatological composition as defined by claim 15, comprising an ethanol solvent.

17. The pharmaceutical/dermatological composition as defined by claim 1, comprising from 70 to 90% by weight with respect to the total weight of the composition of said solvent/cosolvent.

18. The pharmaceutical/dermatological composition as defined by claim 1, comprising a water cosolvent.

19. The pharmaceutical/dermatological composition as defined by claim 18, comprising from 0.01 to 30% by weight of water, with respect to the total weight of the composition.

20. The pharmaceutical/dermatological composition as defined by claim 1, comprising a plasticizing agent selected from among phthalates, triacetates, citrates and mixtures thereof.

21. The pharmaceutical/dermatological composition as defined by claim 20, comprising from 0.01 to 10% by weight of said plasticizing agent, with respect to the total weight of the composition.

22. The pharmaceutical/dermatological composition as defined by claim 1, comprising a film-forming solution or nail varnish.

23. The pharmaceutical/dermatological composition as defined by claim 1, comprising a sprayable solution.

24. The pharmaceutical/dermatological composition as defined by claim 1, comprising a propellant gas.

25. The pharmaceutical/dermatological composition as defined by claim 24, said propellant gas comprising propane, butane, dichlorodifluoromethane, dichlorotetrafluoroethane, octafluorocyclobutane, nitrogen, carbon dioxide, dimethyl ether or mixtures thereof.

26. The pharmaceutical/dermatological composition as defined by claim 24, said propellant gas being in liquid form.

27. The pharmaceutical/dermatological composition as defined by claim 24, comprising from 0.01 to 30% by weight of said propellant gas, with respect to the total weight of the composition.

28. The pharmaceutical/dermatological composition as defined by claim 1, further comprising at least one other active agent selected from among an antibiotic agent, an anti-bacterial agent, a steroidal anti-inflammatory agent, a non-steroidal anti-inflammatory agent, an anti-parasitic agent, an anti-viral agent, an immunosuppressant agent, an agent which is a modulator of nuclear receptors, an anti-fungal agent and mixtures thereof.

29. A regime or regimen for the treatment and/or for the prevention of dermatological pathologies selected from among onychomycosis, green nails, paronychia, acute bacterial paronychia, chronic paronychia, erysipeloid, onychorrexis, gonococcus infection, swimming pool granuloma, larva migrans, leprosy, orf nodule, milkers' nodule, herpetic whitlow, sporotrichosis, syphilis, tuberculosis verrucosa cutis, tularaemia, tungiasis, peri- and subungual warts, herpes zoster, twenty-nail dystrophy and dermatological diseases which affect the nails, psoriasis, pustular psoriasis, alopecia areata, parakeratosis pustulosa, contact dermatosis, Reiter's syndrome, psoriasiform acral dermatitis, lichen planus, idiophatic atrophy of the nails, lichen nitidus, lichen striatus, inflammatory linear verrucous epidermal naevus, pelade, pemphigus, bullosa pemphigoid, acquired epidermolysis bullosa, Darier's disease, pityriasis rubra pilaris, palmoplantar keratoderma, contact eczema, erythema multiforme, scabies, Basex's syndrome, systemic scleroderma, systemic lupus erythematosus, chronic lupus erythematosus and dermatomyositis, comprising administering to a mammal in need of such treatment, a thus effective among of a pharmaceutical/dermatological composition as defined by claim 1.

30. The regime or regimen as defined by claim 29, comprising the treatment and/or the prevention of onychomycosis.
